(19) United States
(12) Patent Application Publication
(10) Pub. No.: US 2023/0190675 A1
(43) Pub. Date: Jun. 22, 2023

(54) VITAMIN A FORMULATION

(71) Applicant: ADISSEO FRANCE S.A.S., ANTONY (FR)

(72) Inventors: Claude ALEMAN, COMMENTRY (FR); Robert HUET, PARIS (FR)

(21) Appl. No.: 17/999,289

(22) PCT Filed: Jun. 4, 2021
(86) PCT No.: PCT/FR2021/051011

§ 371 Date: Nov. 18, 2022

(30) Foreign Application Priority Data
Jun. 5, 2020 (FR) 20/05883

(51) Int. Cl. A61K 31/07; A61K 31/355; A61K 31/593; A61K 31/192; A61K 36/534; A61K 36/537; A61K 36/53

(52) U.S. Cl. CPC A61K 31/07; A61K 31/355; A61K 31/593; A61K 31/192; A61K 36/534; A61K 36/537; A61K 36/53

(57) ABSTRACT

A combination of at least vitamin A and an antioxidant agent having a mixture of carnosic acid and carnosol, the carnosic acid content of the antioxidant agent being 50-98% by weight relative to the weight of the antioxidant agent. A formulation containing such a combination and the use of this combination is also related.

### TECHNICAL FIELD

The disclosure concerns the use of a mixture of carnosic acid and carnosol as an antioxidant agent in formulations of hydrophobic and liposoluble active ingredients.

### BACKGROUND

These molecules, such as vitamins, fatty acids, are very widely used in many technical fields such as the pharmaceutical, cosmetics, agri-food industries, and particularly in the field of animal nutrition. For example, the liposoluble vitamins A, E and D3 are commonly used for the preparation of feed promoting the growth of animals, and in particular livestock.

Their hydrophobic nature and their environmental fragility, particularly thermal and chemical, both during their formulation and their storage, and during their use, make it necessary to preserve them by integrating protective agents and/or by a suitable manufacture, particularly by encapsulation.

The present disclosure is more particularly described below with reference to vitamin A, possibly associated with one or other liposoluble vitamins such as vitamins E and D3 but, of course, its scope is not restricted thereto, and it can be applied to any hydrophobic and liposoluble active substance and any mixture of such substances.

Vitamin A exists in free form but also in other forms, particularly in ester state, the most stable and therefore the most commonly used forms being vitamin A acetate, vitamin A propionate and vitamin A palmitate. In the present text, the term vitamin A will include any one of these forms, particularly free or esterified, as well as any mixture of at least any two of these forms.

Even in its most stable forms, vitamin A is still very sensitive to oxidation resulting from oxidizing agents such as oxygen, temperature, water and light, as well as to the action of acids. In pharmaceutical applications or in animal nutrition, it is thus very quickly degraded as soon as it comes into contact with the first harsh conditions, particularly acidic conditions, of the digestive system, which does not make it a bioavailable form of vitamin A.

In order to best preserve vitamin A, it has long been known to protect it against oxidation by combining it with an antioxidant. Such an agent is generally selected from 3-tert-butyl-4-hydroxy-anisole (BHA), 2,6-di-tert-butyl-4-methyl-phenol (BHT), 6-ethoxy-1,2-dihydroxy-2,2,4-trimethyl-quinoline (ethoxyquinine), 2-tertiobutyl-1,4-dihydroxybenzene or even tocopherol, citric acid or phytic acid and their alkali metal salts, or even ethylenediamine tetraacetic acid (EDTA) and ascorbic acid. Among these, BHT is still the most widely used due to its powerful effectiveness at low cost and its temperature stability. However, it is not devoid of toxicity and is increasingly criticized, suspected of being an endocrine disruptor, as well as being responsible for allergies and increasing the risk of existing tumor development.

Thus, the disclosure aims to replace at least in part, or even totally, the conventionally used antioxidants derived from chemical synthesis and in particular BHT, by a more natural antioxidant in that it can be obtained by means using less chemicals, is of little or no harm and is readily available.

Research has already been undertaken to that regard, particularly for human food. Thus, some plants of the Lamiaceae family known to have an antioxidant effect have been the subject of work and particularly rosemary (whose scientific name is Rosmarinus officinalis).

According to the document R. Inatani et al. Agric. Biol. Chem. (1983) 47 (3), 521, the antioxidant activity of different rosemary leaf extracts is determined. These extracts are obtained using different solvents, n-hexane, dichloromethane and ethanol and their antioxidant activity is measured. From the extracts showing a strong antioxidant activity, fractions are separated, the antioxidant activity of which is measured. Compounds are isolated from the fractions having the strongest activity among which, rosmanol and carnosol have the highest antioxidant power.

The document N. Nakatani et al. Agric. Biol. Chem. (1984) 48 (8), 2081 relates to the isolation of two isomers of rosmanol, epirosmanol and isorosmanol, from a rosemary extract.

The document M. Loussouarn et al. Plant Physiology (2017) 175, 1381 concerns a study on the mechanisms of action of carnosic acid and carnosol as an antioxidant against membrane lipids, in vitro and in vivo.

This work mentions that, depending on the method for measuring the antioxidant power and the tested concentrations, some of the isolated compounds have an activity close to that of BHT or BHA, which may even be similar, or even greater.

According to document CN109452467, vitamin A microcapsules have been described, the core of which includes vitamin A in a content of up to 54% by weight relative to the weight of the microcapsule, mixed with ascorbyl palmitate, carnosic acid and choline or derivative and possibly an oil, and the shell is based on gelatin and polysaccharide(s). The authors carried out comparative aging tests with different microcapsules not meeting the above definition and particularly microcapsules whose core does not comprise choline or derivative, and microcapsules whose core comprises neither ascorbyl palmitate, nor choline or derivative. The results demonstrate that the presence of the combination of ascorbyl palmitate, carnosic acid and choline or derivative is essential, though not sufficient, to give the vitamin A formulation sufficient stability for use in food or animal nutrition.

### SUMMARY

The disclosure offers a solution making it possible to overcome all the problems encountered by existing formulations by providing an antioxidant which has, against vitamin A and the liposoluble vitamins which are possibly associated therewith, a protective effect against oxidation which proves to be much higher than that of BHT or BHA. It is also of natural origin. According to the disclosure, this antioxidant comprises or consists of a mixture of carnosic acid and carnosol, the carnosic acid content of said antioxidant agent being 50-98% by weight relative to the weight of said antioxidant agent.

As will be demonstrated in the examples, the antioxidant power of these extracts is similar to that of BHT or BHA in proportions which are however lower.

Thus, the disclosure provides a combination of at least vitamin A and an antioxidant agent comprising or consisting of a mixture of carnosic acid and carnosol, the carnosic acid content of said antioxidant agent being 50-98% by weight relative to the weight of said antioxidant agent.

### DETAILED DESCRIPTION OF THE DISCLOSURE

According to one implementation of the disclosure, the carnosol content of said antioxidant agent is 2-15% by weight relative to the weight of said antioxidant agent.

The antioxidant agent of the disclosure may consist of said mixture of carnosic acid and carnosol and thus completely replace all other conventionally used antioxidant agents. In this variant, it has been observed that it can be effective at a concentration relative to vitamin A that is much lower than those of common antioxidants such as BHT. It has surprisingly been found that some vehicles potentiate its antioxidant effect, this is the case of some oils, preferably edible oils such as sunflower oil. The mixture of carnosic acid and carnosol can therefore be in powder form or else be present in a vehicle such as an edible oil.

The antioxidant agent of the disclosure may also comprise said mixture of carnosic acid and carnosol. Thus, in addition to this mixture, it may comprise any other antioxidant agent from the list cited above, such as BHT, BHA, EDTA and ascorbic acid, or any compound proving to be antioxidant against vitamin A. Indeed, it has been observed that the incorporation of natural compounds such as polyphenols, or of natural extracts, which are particularly rich in polyphenols, and in particular of a green tea extract, a grape seed extract, or a mixture thereof acts favorably on the antioxidant power of said mixture.

A combination may comprise vitamin A as the only liposoluble active ingredient, but it may also comprise, in addition to vitamin A, one or other liposoluble vitamins such as vitamin E and vitamin D3.

As previously stated, the antioxidant is available from plant sources. The aforementioned mixture of carnosic acid and carnosol may actually be in the form of a Lamiaceae extract and preferably a rosemary, sage or mint extract.

By rosemary, sage or mint extract it should be understood an extract obtained by at least one extraction step; it is also understood that the extract has undergone complementary treatment(s) resulting in the concentration of said extract in some compounds and particularly in carnosic acid and/or in carnosol. The rosemary, sage or mint extracts can be alcoholic or hydroalcoholic extracts or supercritical CO₂ extracts of rosemary, sage or mint leaves, according to conventional techniques well known to those skilled in the art. Extracts meeting the features of the disclosure are also marketed. Advantageously according to the disclosure, rosemary extracts are used.

The Lamiaceae extract may be in powder form or be present in a vehicle such as an edible oil.

According to one implementation of the disclosure, the vitamin A content, and optionally at least one of vitamin E and vitamin D3 contents, in the combination is 75-99% by weight relative to the weight of the combination.

The disclosure also concerns a vitamin A formulation comprising at least one combination as previously defined, a sugar and a gelling agent.

For a beneficial implementation of the disclosure, such a formulation preferably comprises 30-35% by weight of vitamin A and optionally at least one of vitamin E and vitamin D3, 1-10% by weight of said antioxidant agent, 25-30% by weight of a sugar and 23-28% by weight of a gelling agent, the percentages being expressed by weight relative to the weight of the formulation.

A formulation of the disclosure may comprise additive(s) selected from edible oils, crosslinking agents, anti-caking agents, surfactants.

A formulation of the disclosure may be in powder form, for example the particle size of which is preferably in the range of 250 to 350 μm, in the form of microbeads.

It has been unexpectedly observed that, in addition to its antioxidant power, the mixture of carnosic acid and carnosol has a chelating power capable then of reinforcing the protection of vitamin A against deleterious cations which would be present in the formulation ingredients. This is an additional advantage compared to conventional antioxidants and particularly BHT.

As will be presented in more detail below, a formulation of the disclosure can be obtained by double emulsion or by atomization of an emulsion; such an emulsion preferably comprises at least 10 to 22% by weight of vitamin A and optionally at least one of vitamin E and vitamin D3, 0.5 to 5% by weight of an antioxidant of the disclosure, 13 to 20% by weight of said sugar and 12 to 18% by weight of said gelling agent.

The vitamin A formulations of the disclosure can be prepared by known methods such as those described in EP285682A or WO2011/070300A.

As detailed in the document EP285682A, an emulsion containing vitamin A, water, an antioxidant, gelatin and a sugar is prepared and converted into droplets by spraying. These droplets are then brought into contact individually with a cellulose powder having well-defined characteristics until hardening of the droplets. The droplets thus hardened are then separated from the cellulose powder by sieving. The recovered droplets are subsequently dried, then subjected to a heating operation in order to ensure crosslinking of the gelatin by reaction of the amino groups of the gelatin with the reducing functions of the sugar.

It is also possible to manufacture a formulation according to the method described by the document WO2011/070300A according to the following steps:
an oil-in-water emulsion is prepared comprising, in weight percent relative to the total weight of said emulsion:
8 to 20%, preferably 10 to 15% of at least one protein,
5 to 15%, preferably 8 to 12% of at least one sugar,
0.5 to 3%, preferably 2 to 3% of at least one inorganic salt,
0.68-5% of an antioxidant of the disclosure,
10 to 22%, preferably 15 to 20% of at least one liposoluble active ingredient and in particular vitamin A in oily form and/or dissolved in a food oil,
qs % of water,
particles are formed in substantially spherical shape by dispersing the oil-in-water emulsion obtained at the end of step a) in a fluid,
at least one protein crosslinking agent is added to the dispersion,
the active ingredients are recovered in the substantially spherical shape.

According to particular implementations of this method:
the protein(s) are selected from gelatin, casein or caseinate, soy or corn proteins;
the sugar(s) are selected from polyols, monosaccharides, disaccharides, glucose and fructose syrups and maltodextrins;
the inorganic salt(s) are selected from sodium, potassium, lithium salts of mono-, di- and tri-phosphoric acid or polyphosphoric acid, preferably Na₂HPO₄.

This preparation method can also comprise a preliminary step during which vitamin A is dissolved in a food oil such as peanut, sunflower, rapeseed, corn, soybean, palm or cod liver oil.

The disclosure also concerns a feed or fodder for animal nutrition comprising a vitamin A formulation as described above in any one of its variants.

The disclosure further relates to a use of a mixture of carnosic acid and carnosol as or in an antioxidant agent in a formulation of vitamin A, and optionally of at least one of vitamin E and vitamin D3. Preferably, the carnosic acid content of said antioxidant agent is 50-98%, and more preferably, the carnosol content of said antioxidant agent is 2-15% by weight relative to the weight of said antioxidant agent.

The disclosure is hereinafter illustrated in the following examples, describing in detail some implementations and demonstrating its performance.

### Examples

The antioxidant power measurement methods used in the examples for antioxidant and vitamin A (as vitamin A acetate) mixtures are shown below.

1) Dynamic oxidation test by DSC (Differential Scanning calorimeter)

It is carried out under oxygen flow (50 ml/min) then the temperature is raised to a temperature above the melting point of vitamin A (i.e. above 60° C.).

The oxidation corresponds to an exothermic phenomenon which occurs at a given temperature. For BHT, it occurs at 153° C. The higher the oxidation temperature, the higher the antioxidant power.

All the samples having a temperature greater than or equal to 120° C. will be retained for a much longer Oxipres test.

2) Oxipres test:

This test consists of putting the samples under O₂ pressure at a given temperature. The method allows determining the kinetics of oxygen consumption, expressed by a variation of O₂ (ΔP in bars), this variation being all the smaller as the antioxidant power is high.

### Example 1: Preparation of Samples to be Tested

The samples 2-6 are prepared by mixing vitamin A acetate and an antioxidant present in the combination in a content of 20% by weight.

They are then tested according to the evaluation models described above, as well as a sample 1 of vitamin A acetate (without antioxidant).

### Example 2: Results of the DSC 1) and Oxipres Tests on a Binary Mixture

The test results for the samples 1-6 are shown in the following table. The mixture of carnosic acid/carnosol is abbreviated AC/c.

TABLE 1

| | | | Oxipres |
|---|---|---|---|
| | | DSC DYN | Time (h) |
| Sample No | Antioxidant | Onset (° C.) | ΔP (Bars) |
| 1 | None | 60° C. | 0 |
| | | | 5.0 |
| 2 | BHT | 153.60 | >168 |
| | | | ΔP = 1.8 |
| 3 | α Tocopherol | 127.00 | >168 |
| | | | ΔP = 2.25 |
| 4 | AC/c + | 130.00 | >168 |
| | green tea extract and | | ΔP = 1.2 |
| | grape seed extract* | | |
| 5 | AC 85%/c 15% | 132.00 | >168 |
| | in rosemary extract form | | ΔP = 1.2 |
| 6 | AC 85%/c 15% | 143.00 | |
| | in sunflower oil 4% | | |

*the mixture of AC/c + green tea extract and grape seed extract is marketed by LAYN under the reference TruGro ®11136-19080504.

It is observed that the power of the antioxidants of the samples 4-6 illustrating the disclosure is always higher than that of α-tocopherol in the two evaluation tests, and is close to, or even higher in the Oxipres evaluation test than, that of BHT. It is also noted that this power is expressed at very low contents of the mixture of AC/c, the so result of the sample 6 is particularly interesting.

1. A combination of at least vitamin A and an antioxidant agent, the combination comprising: a mixture of carnosic acid and carnosol, the carnosic acid content of said antioxidant agent being 50-98% by weight relative to the weight of said antioxidant agent.

2. The combination according to claim 1, wherein the carnosol content of said antioxidant agent is 2-15% by weight relative to the weight of said antioxidant agent.

3. The combination according to claim 1, wherein the antioxidant further comprises at least one of BHT, BHA, EDTA and ascorbic acid.

4. The combination according to claim 1, wherein the antioxidant further comprises polyphenols.

5. The combination according to claim 1, of vitamin A and at least one vitamin selected from vitamin E and vitamin D3.

6. The combination according to claim 1, wherein the mixture of carnosic acid and carnosol is in the form of a Lamiaceae extract and a rosemary, sage, or mint extract.

7. The combination according to claim 6, wherein the rosemary extract is selected from alcoholic or hydroalcoholic extracts and supercritical CO₂ extracts of rosemary leaves.

8. The combination according to claim 1, wherein the said mixture or said Lamiaceae extract is in powder form or is present in a vehicle such as an edible oil.

9. The combination according to claim 1, wherein the vitamin A content, and at least one of vitamin E and vitamin D3 contents, is 75-99% by weight relative to the weight of the combination.

10. A formulation of at least vitamin A comprising at least one combination according to claim 1, a sugar, and a gelling agent.

11. The formulation according to claim 10, further comprising 30-35% by weight of vitamin A and optionally at least one of vitamin E and vitamin D3, 1-10% by weight of said antioxidant agent, 25-30% by weight of a sugar and 23-28% by weight of a gelling agent.

12. The formulation according to claim 10, wherein the formulation comprises additive(s) selected from edible oils, crosslinking agents, anti-caking agents, and surfactants.

13. The formulation according to claim 10, wherein the formulation is obtained by double emulsion or by atomization of an emulsion comprising at least 10-22% by weight of vitamin A and optionally at least one of vitamin E and vitamin D3, 0.5-5% by weight of said antioxidant agent, 13-20% by weight of said sugar and 12-18% by weight of said gelling agent.

14. The formulation according to claim 10, which is in powder form.

15. A feed or fodder for animal nutrition comprising a vitamin A formulation according to claim 10.

16. A use of a mixture of carnosic acid and carnosol as or in an antioxidant agent in a vitamin A formulation, and at least one of vitamin E and vitamin D3.
